(19) United States
(12) Patent Application Publication
(10) Pub. No.: US 2015/0297600 A1
(43) Pub. Date: Oct. 22, 2015

(54) Method Of Achieving Increased Systemic Availability of Methotrexate

(71) Applicant: Antares Pharma, Inc., Ewing, NJ (US)

(72) Inventors: Peter L. SADOWSKI, Woodbury, MN (US); Kaushik J. DAVE, Edison, NJ (US); Paul K. WOTTON, Newtown, PA (US); Hermanus L. JOOSTE, Wayne, PA (US)

(21) Appl. No.: 14/688,061

(22) Filed: Apr. 16, 2015

Related U.S. Application Data
(60) Provisional application No. 61/980,376, filed on Apr. 16, 2014.

(51) Int. Cl. A61K 31/519; A61M 5/00

(52) U.S. Cl. CPC A61K 31/519; A61M 5/002

(57) ABSTRACT

Determining that a patient in need of methotrexate requires more systemically available methotrexate than can be obtained by an orally administered dose of methotrexate; and subcutaneously administering a dose of methotrexate to the patient.

### CROSS REFERENCE TO RELATED APPLICATIONS

The present application claims priority to pending U.S. Provisional Patent Application No. 61/980,376 filed Apr. 16, 2014, which is incorporated by reference herein for all purposes.

### FIELD OF THE DISCLOSURE

The present disclosure relates to methods of determining dosages of compositions used in injection devices, in view of known oral doses of the same compositions.

### BACKGROUND INFORMATION

Hazardous agents, such as cytotoxic agents, have been useful in managing and treating a number of diseases such as rheumatoid arthritis (and other autoimmune diseases), juvenile rheumatoid arthritis, psoriatic arthritis, systemic lupus erythematosus, steroid-resistant polymyositis or dermatomyositis, Wegener's granulomatosis, polyarteritis nodosa, and some forms of vasculitis. Hazardous agents tend to exhibit side effects, however, that are harmful or toxic to the subject. Many of these side effects occur when hazardous agents are administered orally, but the oral form is generally the preferred method of delivery of these agents due to its ease of use.

In addition to increased toxicity, variable and reduced bioavailability has been observed for some hazardous agents, such as methotrexate, that are orally administered. These limitations are particularly demonstrated when the oral dosing is escalated beyond 15 mg per dose. It has been suggested that with parenteral administration, such as by injection, more predictable, reproducible and complete bioavailability along with better therapeutic results could be achieved, particularly at higher dosages.

Because of the large number of precautions that must be learned and followed in order to safely inject a hazardous agent, and to inject a hazardous agent in therapeutically accurate amounts, it is presently thought that it is not practical for hazardous agents, and particularly methotrexate, to be self-injected by a patient outside of a clinical setting or without the assistance of a health care provider.

### SUMMARY

In an embodiment, disclosed herein is a method of determining that a patient in need of methotrexate requires more systemically available methotrexate than can be obtained by an orally administered dose of methotrexate and subcutaneously administering a dose of methotrexate to the patient. Kits relating to the same are also disclosed.

### BRIEF DESCRIPTION OF THE DRAWINGS

These and other objects, features and advantages of the invention will be apparent from a consideration of the following non-limiting detailed description considered in conjunction with the drawing figures, in which:

FIG. 1 is a graph illustrating an equation developed to determine the correlation between an oral dose and an equivalent jet-injected dose of a medicament.

### DETAILED DESCRIPTION

Various embodiments of the present invention are described more fully below. Some but not all embodiments of the present invention are shown. Indeed, various embodiments of the invention may be embodied in many different forms and should not be construed as limited to the embodiments expressly described. Like numbers refer to like elements throughout. The singular forms “a,” “an,” and “the” include the singular and plural unless the context clearly dictates otherwise.

Methods

Most drugs are designed to go from injectable to oral. Companies usually develop an oral drug after all the early work has been done in an injectable, which establishes the drug's toxicity etc. Oral availability is usually a fraction of the injectable bioavailability. The present invention turns this practice on its head.

In an embodiment, to improve, among other things, the accuracy, efficacy, safety, and efficiency of dosage of therapeutic agents, the correlation between oral and equivalent injectable dosage must be understood. The present disclosure addresses this need.

In an embodiment, disclosed herein is a method of administering a medicament to a subject, comprising identifying a suitable oral dose of a medicament, converting the oral dose of the medicament to an equivalent parenteral (e.g., subcutaneous, intravenous, intramuscular, or intradermal) dose of the medicament, and administering the equivalent dose of the medicament to the subject. In at least one embodiment, a medicament in accordance with the present invention may be administered via a variety of routes of administration. In at least one embodiment, a medicament in accordance with the present invention may be administered via any combinations of oral, rectal, nasal, pulmonary, epidural, ocular, otic, intra-arterial, intracardiac, intracerebroventricular, intradermal, intravenous, intramuscular, intraperitoneal, intraosseous, intrathecal, intravesical, subcutaneous, topical, transdermal, transmucosal, sublingual, buccal, vaginal, and inhalational routes of administration.

In at least one embodiment of a method of administering a medicament in accordance with the present invention, the method may include transitioning a subject in need of such treatment from an oral route of administration of the medicament to a parenteral route of administration of the medicament. In at least one embodiment of a method of administering a medicament in accordance with the present invention, the method may include evaluation of the effect of a particular route of administration of the medicament on the systemic availability of the medicament. In at least one embodiment of a method of orally administering a medicament in accordance with the present invention, the method may include evaluation of the effect of one or more of the factors that are unique to gastrointestinal track on the systemic availability of the medicament. In at least one embodiment of a method of orally administering a medicament in accordance with the present invention, the method may include evaluation of the effect of one or more of medicament absorption factors, including, but not limited to, intestine contents (such as food), lumen pH, intestinal motility, intestinal digestion, presence of specific transport mechanisms, and disease states and/or evidence of damage to the intestine.

In at least one embodiment of a method of administering a medicament by injection in accordance with the present invention, the method may include evaluation of the effect of one or more of factors unique to injection site and/or the physical properties of the method of injection.

In at least one embodiment of a method of administering a medicament by injection in accordance with the present invention, the method may include evaluation of the effect of one or more medicament absorption factors on systemic availability of the injected medicament, including, but not limited to, injection into muscle tissue, injection into subcutaneous tissue, rate at which the medicament is delivered, force of injection, concentration of the medicament in a defined unit of volume and area of dispersion of the medicament once injected.

In some embodiments, the present invention provides a method of deriving a conversion factor for facilitating calculation of a correct dose of a medicament for administration to a subject by injection route based on an established efficacy of an oral dose of the medicament, and/or a presumed efficacy of an oral dose of the medicament, and/or a desired systemic medicament concentration. In at least one embodiment of the method of deriving a conversion factor for facilitating calculation of a correct injectable dose of a medicament, the method comprises determining systemic availability of the medicament over several injectable medicament concentrations. In at least one embodiment of the method of deriving a conversion factor for facilitating calculation of a correct injectable dose of a medicament, the method comprises determining systemic availability of the medicament over several injectable medicament concentrations, wherein the medicament concentrations are selected from a range of medicament doses having therapeutic significance. In at least one embodiment of the method of deriving a conversion factor for facilitating calculation of a correct injectable dose of a medicament, the method includes determining the systemic availability of the medicament at various concentrations when the medicament is given by injection to a subject. In at least one embodiment of the method of deriving a conversion factor for facilitating calculation of a correct injectable dose of a medicament, bioavailability of the medicament is determined for a particular route of administration. In at least one embodiment of the method of deriving a conversion factor for facilitating calculation of a correct injectable dose of a medicament, bioavailability of the medicament is determined for administration by injection with a particular injection system. In at least one embodiment of the method of deriving a conversion factor for facilitating calculation of a correct injectable dose of a medicament, wherein bioavailability of the medicament is determined for administration by injection with a particular injection system, the method further includes evaluation of injection system performance. In at least one embodiment of the method of deriving a conversion factor for facilitating calculation of a correct injectable dose of a medicament, wherein bioavailability of the medicament is determined for administration by injection with a particular injection system, the method further includes evaluation of injection system performance through evaluation of one or more injection system factors, including, but not limited to, needle gauge, needle insertion length, and injection rate.

In at least one embodiment of the method of deriving a conversion factor for facilitating calculation of a correct injectable dose of a medicament, the method includes (i) determining systemic availability of a medicament at various concentrations when the medicament is given by injection to a subject; and (ii) determining systemic availability of the medicament at various concentrations when the medicament is administered orally to a subject.

In at least one embodiment of the method of deriving a conversion factor for facilitating calculation of a correct injectable dose of a medicament, the method includes (i) determining systemic availability of a medicament at various concentrations when the medicament is administered by injection to a subject; (ii) determining systemic availability of the medicament at various concentrations when the medicament is administered orally to a subject; and (iii) a conversion algorithm is created based on the systemic availability of the medicament from obtained from the injection and oral administrations of the medicament at the various concentrations, whereby to the conversion algorithm allows/facilitates determination of the correct dose of the medicament.

In an embodiment, an equivalent peritoneal dose of the medicament is further converted into an administrable dose. In an embodiment, an administrable dose is based on the commercially available dose of the medicament that is closest in value (e.g., mg/ml) to the equivalent dose of the medicament.

In one embodiment, the oral dose is converted to an equivalent subcutaneous dose by providing an algorithm and applying the algorithm to the oral dose. Optionally, the algorithm can be provided on a computer-readable medium. In an embodiment, the corresponding subcutaneous dose is obtained by multiplying the oral dose of the medicament by about 0.6101 and adding about 2.9274 to the product, thereby converting the oral dose to an equivalent subcutaneous dose. In an embodiment, more simply, the corresponding subcutaneous dose is obtained by multiplying the oral dose of the medicament by about 0.6 and adding about 3 to the product, thereby converting the oral dose to an equivalent subcutaneous dose. In an embodiment, a method further comprises the step of obtaining an administrable dose based on the equivalent subcutaneous dose. In an embodiment, an administrable dose is based on one or more commercially available doses. In an embodiment, an administrable dose is based on the commercially-available dose that is closest to the equivalent subcutaneous dose. However, as shown in Table 1 below, the lowest dose is typically 7.5 mg subcutaneous, which matches oral dose. Accordingly, one of skill in the art understands that through using the algorithm one can round to the nearest administrable dose.

In an embodiment, the subcutaneous dose is calculated for delivery by a jet injector. In an embodiment, the subcutaneous dose is calculated for delivery by a needle-assisted jet injector. Such injectors are described in greater detail elsewhere herein.

In one embodiment, a method is provided for determining the amount of a medicament to administer to a subject subcutaneously, comprising identifying a suitable oral dose of a medicament, then converting the oral dose of the medicament to an equivalent subcutaneous dose of the medicament by applying an algorithm designed to correlate the oral dose of a medicament to a corresponding equivalent subcutaneous dose of the medicament. In an embodiment, an equivalent subcutaneous dose of a medicament is a dose of the medicament that, when administered subcutaneously to a subject, results in bioequivalence with the orally administered dose. In an embodiment, an equivalent subcutaneous dose of a medicament is a dose of the medicament that, when administered subcutaneously to a subject, is therapeutically equivalent to the orally administered dose. In an embodiment, an equivalent subcutaneous dose of a medicament is a dose of the medicament that, when administered subcutaneously to a subject, is substantially bioequivalent to the orally administered dose. In an embodiment, the medicament comprises methotrexate.

In some embodiments, an equivalent dose is calculated based on bioavailability of the therapeutic agent. Such methods can be found in U.S. Patent Application Publication 2012/0157965, U.S. patent application Ser. No. 13/564,693, and PCT/US2012/049235, each of which is incorporated herein by reference in its entirety.

In some embodiments, an equivalent dose of a therapeutic agent is determined by assessing the bioavailability of the therapeutic agent. In some embodiments, the bioavailability of a therapeutic agent can be approximated to a known or desired level by selecting one or more factors in the configuration of a jet injector, to maintain bioequivalence for the therapeutic agent. In an embodiment, bioequivalence can be measured using means known in the art to measure plasma levels to determine the rate and extent of absorption of the therapeutic agent and determining the extent of absorption. One such measure known in the art is the determination of area under the concentration curve, the AUC, which is directly proportional to the amount of therapeutic agent in the patients's blood. Another such measure is the determination of the peak serum concentration of therapeutic agent, the Cₘₐₓ.

In an embodiment, bioequivalence may be established if a therapeutic agent injected via an injector according to the present disclosure reaches the site of absorption in similar times and is absorbed to the same extent as if the hazardous agent had been introduced to the subject via other known routes of administration. In some embodiments, an equivalent dose of a therapeutic agent is determined by a method comprising assessing the indirect actions of the therapeutic agent after administration.

In some embodiments, also disclosed herein are methods of converting an oral dose of a medicament to an equivalent subcutaneous dose of the medicament, comprising identifying a suitable oral dose of the medicament for a subject, providing an algorithm to convert the oral dose to an equivalent subcutaneous dose, and applying the algorithm to the oral dose, thereby converting the oral dose to an equivalent subcutaneous dose, wherein a suitable oral dose of the medicament is a therapeutically effective amount of the medicament. In some embodiments, the methods include a determination of bioavailability, as described in detail elsewhere herein, based on a specific medicament and/or route of administration. In some embodiments, the medicament comprises methotrexate.

In some embodiments, also disclosed herein are methods of converting an oral dose of a medicament to an equivalent subcutaneous dose of the medicament, in view of a specific injector to be used for administration of the subcutaneous dose, comprising identifying a suitable oral dose of the medicament for a subject, identifying a suitable injector device for administering the subcutaneous dose of the medicament, providing an algorithm to convert the oral dose to an equivalent subcutaneous dose to be delivered by the injector device, and applying the algorithm to the oral dose, thereby converting the oral dose to an equivalent subcutaneous dose, wherein a suitable oral dose of the medicament is a therapeutically effective amount of the medicament. Such injectors are described in and encompassed by U.S. Patent Application Publication 2012/0157965, U.S. patent application Ser. No. 13/564,693, International application PCT/US2012/049235, and U.S. Pat. Nos. 6,746,429, 7,744,582, 7,776,015, and 8,021,335, each of which is incorporated herein by reference in its entirety. In some embodiments, the methods include a determination of therapeutic equivalence, as described in detail elsewhere herein, based on a specific injector device. In some embodiments, the methods include a determination of bioavailability, as described in detail elsewhere herein, based on a specific injector device. In some embodiments, the medicament comprises methotrexate and the injector device comprises a needle-assisted injector device.

Therapeutic Agents

In an embodiment, a therapeutic agent, also referred to herein as a medicament, is methotrexate. Other therapeutic agents include, but are not limited to, rifampin, sulfonamides, chloramphenicol, fluoroquinolones, aminopenicilin, natural penicillin, ampicillin, cloxacilin, isoxicam, diclofenac, ketoprofen, acetylsalicylate, meloxicam, tenoxicam, vitamin B6, vitamin B12, and vitamin K, and combinations thereof.

In an embodiment, a dose of a therapeutic agent is identified as a suitable dose. In an embodiment, a suitable dose of a therapeutic agent is an amount of therapeutic agent understood and/or believed to be an amount sufficient to bring about a desired effect in a subject intended to receive the therapeutic agent. In an embodiment, a suitable dose of a therapeutic agent is a therapeutically effective amount of the therapeutic agent. It will be understood by the skilled artisan, when armed with the disclosure encompassed herein, that additional factors may need to be considered for a selected therapeutic agent, including, but not limited to, whether there are any active or passive transport mechanisms that must be taken into account, whether the locus of injection affects the rate and/or degree of uptake of the therapeutic agent, whether injection of the therapeutic agent can affect the activity or availability of the therapeutic agent, and whether the concentration or form of the therapeutic agent can affect the uptake and/or bioavailability of therapeutic agent differently via injection than via oral administration.

In one embodiment, a therapeutic agent is provided in a therapeutically effective amount. In an aspect, an oral dose that forms the basis for the determination of an equivalent subcutaneous dose is a dose in a therapeutically effective amount. In an embodiment, a therapeutically effective amount of a therapeutic agent is a suitable dose. “Therapeutically effective amount” refers to the amount of a therapeutic agent that, when administered to a subject for treating a disease or disorder, or at least one of the clinical symptoms of a disease or disorder, is sufficient to affect such treatment of the disease, disorder, or symptom. The therapeutically effective amount may vary depending, for example, on the compound, the disease, disorder, and/or symptoms of the disease, severity of the disease or disorder, and/or symptoms of the disease or disorder, the age, weight, and/or health of the patient to be treated, and the judgment of the prescribing physician. A therapeutically effective amount may be ascertained by those skilled in the art or capable of determination by routine experimentation. In an embodiment, a therapeutically effective amount is determined on the same clinical outcome being defined by an oral dose and by a dose administered via an alternate route of administration (e.g., subcutaneous).

In some embodiments, the therapeutic agent is methotrexate and/or one or more derivatives of methotrexate. Methotrexate and derivatives of methotrexate, as well as the pharmacokinetics of methotrexate, administered both orally an injected, are described in greater detail in U.S. Patent Application Publication 2012/0157965, incorporated herein by reference in its entirety. In some embodiments, encompassed are methotrexate and/or derivatives of methotrexate and/or pharmaceutically acceptable salts, solvates, hydrates, oxides and N-oxides thereof, are injected. In some embodiments, the present disclosure relates to the methotrexate and/or derivatives of methotrexate and one or more pharmaceutically acceptable excipients. In some embodiments, the present disclosure relates to a pharmaceutically acceptable salt of methotrexate and/or derivatives of methotrexate. In some embodiments, the present disclosure relates to pharmaceutical compositions comprising methotrexate and a pharmaceutically acceptable excipient.

Injection

In some embodiments, a parenteral dose is a dose that is delivered to a subject by way of an injector device. In some embodiments, a subcutaneous, intravenous, intramuscular, or intradermal dose is a dose that is delivered to a subject by way of an injector device. In some embodiments, a subcutaneous dose is a dose that is delivered to a subject by way of a jet injector device. In some embodiments, a jet injector device does not comprise a needle. In some embodiments, a jet injector device does not comprise the use of a needle. In some embodiments, a jet injector device is a needle-assisted jet injector, also referred to as a high pressure auto-injector. In some embodiments, a jet injector device is a needle assisted jet injector comprising a syringe.

In some embodiments, the present disclosure encompasses the conversion of a parenterally administered medicament dose to an equivalent dose of the therapeutic agent for subcutaneous injection via a jet injector. In some embodiments, the jet injector is a needle-assisted jet injector.

In some embodiments, the present disclosure relates to the conversion of an oral dose of a therapeutic agent to an equivalent dose of the therapeutic agent for subcutaneous injection via a jet injector. In some embodiments, the jet injector is a needle-assisted jet injector. In some embodiments, the jet injector is a needle-free jet injector.

Non-limiting examples of suitable injectors and injection methods can be found in U.S. Patent Application Publication 2012/0157965, U.S. patent application Ser. No. 13/564,693, International application PCT/US2012/049235, and U.S. Pat. Nos. 6,746,429, 7,744,582, 7,776,015, and 8,021,335, each of which is incorporated herein by reference in its entirety. As will be understood by the skilled artisan, when armed with the disclosure encompassed herein, one or multiple factors may be used to alter the delivery of a medicament via an injector, and the subsequent bioavailability and/or therapeutic effect, including, but not limited to, the size of the injected dose, the formulation of the injected dose, the speed of the injection, the method of injection, the use of a needle to assist injection, the depth of the injector, the depth of the needle, and the site of injection, among other factors.

In an embodiment, a powered injector may be used. In an embodiment, an injector encompassed by the present disclosure uses an energy source that produces moderate to high pressure in the medicament chamber so that a medicament contained in the medicament chamber is fired at a fast speed and is completely injected into a subject in less than about 5 seconds. In another embodiment, the powered injector completely delivers the dose into a subject at a rate that is at least about twice as fast as the comparator injector or delivery system. Other embodiments of the powered injectors are jet injectors, which can be needle-assisted or needle-free jet injectors. Jet injector embodiments can be configured to have an energy source selected to produce a high pressure in the medicament chamber to eject the medicament with sufficient pressure, force, and speed to exit the injector as a fluid jet.

In some embodiments, it has been unexpectedly found that oral doses of methotrexate do not provide increased systemic availability (e.g., bioavailability) of methotrexate for oral doses that are equal to or greater than 15 mg. In other words, the plasma level of methotrexate does not increase in some or all patients as the oral dose of methotrexate increases beyond 15 mg. Accordingly, the blood plasma levels for such patients receiving oral doses of 15 mg, 20 mg, 25 mg, or more may all exhibit the same plateau or saturated blood plasma concentration of methotrexate. Therefore, in patients identified wherein the patient requires greater systemic availability of methotrexate than can be obtained by an oral dose of methotrexate, subcutaneous administered methotrexate can be provided to such patients. In such embodiments, the dose of methotrexate administered subcutaneously can be a dose as described herein. In other embodiments, the subcutaneous dose can be a weekly dose selected from 10 mg, 15 mg., 20 mg, or 25 mg The subcutaneously administered methotrexate may be administered by a hand-powered syringe or a needle-assisted powered injector or needle-free powered injector.

In some embodiments, a kit is provided for administration of a medicament to a subject in need thereof, comprising a jet injector device as encompassed herein for administration (e.g., subcutaneous) of the medicament to the subject and instructions for identifying, e.g., by monitoring blood plasma levels, a patient in need of greater systemic availability of methotrexate than can be obtained by an oral dose of methotrexate and/or instructions for converting an oral dose of the medicament to an equivalent subcutaneous, intravenous, intradermal, or intramuscular dose of the medicament, as described and encompassed herein, for use in conjunction with the jet injector.

### EXPERIMENTAL EXAMPLES

### Example 1

An algorithm was developed to correlate a known oral dose of methotrexate to a jet injector dose. Table 1 illustrates the calculated correlation between oral doses and needle-assisted jet injector doses of methotrexate. The second column illustrates the precise subcutaneous dose calculated based on the algorithm, described in detail below. The third column represents the administrable dose, based on commercially available doses, closest in value to the precise calculated dose.

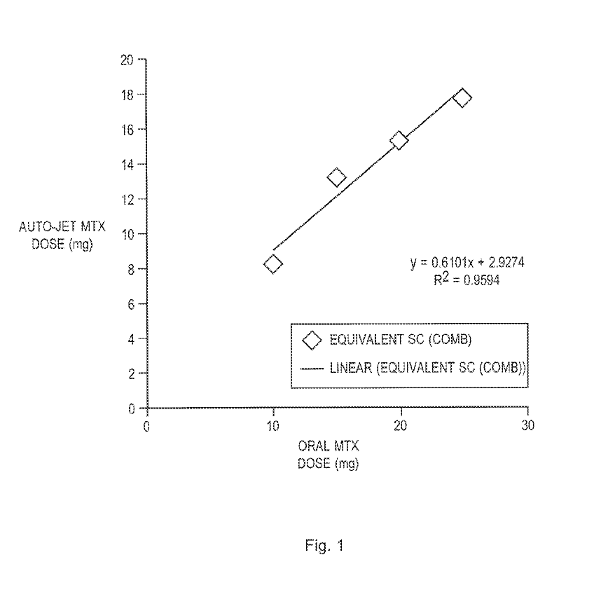

Based on the data set forth in table 1, a line equation of y=mx+b was developed to correlate an oral dose of methotrexate to an equivalent dose for use with a jet injector. In the present equation, illustrated by the graph shown in FIG. 1, the slope, m, was determined to be 0.6101 and a y-intercept, b, to be 2.9274. The correlation coefficient of the line, R², is 0.9594. As shown in FIG. 1, the x value represents the known oral dose of methotrexate, and the y value, which can be obtained using the algorithm described herein, will provide the corresponding jet injector dose.

TABLE 1
Oral to jet injector dose conversion for methotrexate.

| | Precise | Selected |
|---|---|---|
| Oral Dose | Subcutaneous | Administrable |
| (mg) | Dose (mg) | Dose (mg) |
| 7.5 | 7.50315 | 7.5 |
| 10 | 9.0284 | 10 |
| 12.5 | 10.55365 | 10 |
| 15 | 12.0789 | 10 |
| 17.5 | 13.60415 | 15 |
| 20 | 15.1294 | 15 |
| 22.5 | 16.65465 | 15 |
| 25 | 18.1799 | 20 |
| 27.5 | 19.70515 | 20 |
| 30 | 21.2304 | 20 |

### Example 2

A multicenter, three-way crossover study was conducted, wherein patients greater than or equal to 18 years old with adult RA undergoing treatment with MTX for three months or more were assigned to receive one of four dose levels of OTREXUP™ (subcutaneous methotrexate), 10 mg, 15 mg, 20 mg, and 25 mg weekly in a random sequence of three treatments: oral, subcutaneous into the abdomen and subcutaneous into the thigh. For 24 hours after the administration of each treatment, blood samples were collected to measure drug levels and injection sites were assessed. Forty-seven patients completed the study and the results showed that the systemic availability of methotrexate following oral dosing plateaus at 15 mg and greater. Following administration of OTREXUP™, the systemic availability increased proportionally at every dose, which extended the range of exposure compared to patients receiving oral therapy. No unexpected adverse events were noted for either formulation in this short term study and higher systemic methotrexate exposure was not associated with increases in adverse events.

The study results show that plasma levels of oral dosed methotrexate are no greater for 20 mg or 25 mg doses than for 15 mg doses. In view of the foregoing, a patient that fails to respond to 15 mg of methotrexate orally, switching to a subcutaneous regimen rather than continue to raise the oral dose may be more effective.

Each and every reference herein is incorporated by reference in its entirety. The entire disclosure of International Application No. PCT/US2014/024530, U.S. patent application Ser. No. 14/678,449 and U.S. provisional patent application Nos. 61/778,398 are hereby incorporated herein by reference thereto as if fully set forth herein. The term “about,” as used herein, should generally be understood to refer to both the corresponding number and a range of numbers. Moreover, all numerical ranges herein should be understood to include each whole integer within the range.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

What is claimed:

1. A method of achieving increased systemic availability of methotrexate comprising:
(a) determining that a patient in need of methotrexate requires more systemically available methotrexate than can be obtained by an orally administered dose of methotrexate;
and,
(b) subcutaneously administering a dose of methotrexate which provides more systemically available methotrexate than could be obtained by the orally administered dose of methotrexate.

2. The method of claim 1, wherein the orally administered dose is 15 mg of methotrexate.

3. The method of claim 1, wherein the orally administered dose is 20 mg of methotrexate.

4. The method of claim 1, wherein the orally administered dose is 25 mg of methotrexate.

5. The method of claim 1, wherein the dose of methotrexate is administered weekly.

6. The method of claim 5, wherein the dose is 10 mg.

7. The method of claim 5, wherein the dose is 15 mg.

8. The method of claim 5, wherein the dose is 20 mg.

9. The method of claim 5, wherein the dose is 25 mg.

10. A kit for administration of a medicament to a subject in need thereof, the kit comprising:
(a) instructions for identifying a subject in need of an oral dose of methotrexate wherein the patient requires more systemically available methotrexate than can be obtained by an orally administered dose of methotrexate; and,
(b) an injector device for subcutaneous administration of methotrexate to the subject.

11. The kit of claim 10, wherein the injector device is a hand-powered syringe.

12. The kit of claim 10, wherein the injector device is a powered injector.

13. The kit of claim 10, wherein the injector device is a jet injector.

14. The kit of claim 10, wherein the oral dose is 10 mg.

15. The kit of claim 10, wherein the oral dose is 15 mg.

16. The kit of claim 10, wherein the oral dose is 20 mg.

17. The kit of claim 10, wherein the oral dose is 25 mg.
